(19) United States
(12) Patent Application Publication
(10) Pub. No.: US 2025/0107724 A1
(43) Pub. Date: Apr. 3, 2025

(54) SYSTEMS AND METHODS FOR VALIDATING MAGNETIC RESONANCE IMAGING (MRI) MACHINES AND MRI DATA

(71) Applicant: PreOperative Performance Inc., Toronto (CA)

(72) Inventor: Fergal KERINS, Toronto (CA)

(21) Appl. No.: 18/905,081

(22) Filed: Oct. 2, 2024

Related U.S. Application Data
(60) Provisional application No. 63/542,260, filed on Oct. 3, 2023.

(51) Int. Cl. A61B 5/055; G01R 33/563; G01R 33/58

(52) U.S. Cl. CPC A61B 5/055; G01R 33/56341; G01R 33/58

(57) ABSTRACT

A computer-implemented method is provided for the purposes of validating patient data obtained from a magnetic resonance imaging (MRI) machine. The method involves receiving phantom data acquired from scanning a phantom, such as a diffusion tensor imaging (DTI) phantom, designed for the purposes of validating accuracy of in-vivo measurements, such as DTI relevant metrics, across time and vendor, and analyzing the phantom data to generate metrics for assessing MRI performance. Optionally, the MRI machine may be identified as a validated machine as part of the method by comparing the generated MRI metrics with previous metrics stored in a data library.

### RELATED APPLICATIONS

This application claims priority from U.S. Provisional Patent Application No. 63/542,260 filed on Oct. 3, 2023 entitled “SYSTEMS AND METHODS FOR VALIDATING MAGNETIC RESONANCE IMAGING (MRI) MACHINES AND MRI DATA”. This application claims the benefit under 35 USC § 119 of U.S. Provisional Patent Application No. 63/542,260 filed on Oct. 3, 2023 entitled “SYSTEMS AND METHODS FOR VALIDATING MAGNETIC RESONANCE IMAGING (MRI) MACHINES AND MRI DATA”, which is incorporated herein by reference in its entirety.

### TECHNICAL FIELD

The present disclosure relates generally to magnetic resonance imaging (MRI), and in particular to systems and methods for analyzing and validating diffusion weighted imaging (DWI) data, including diffusion tensor imaging (DTI) data. Particular embodiments have example applications for supporting clinical benefit findings in clinical trials and medical device development.

### BACKGROUND

In the field of medicine, diagnostic imaging procedures such as Magnetic Resonance Imaging (MR or MRI) are commonly used to identify certain disease conditions and pathologies affecting organs like the brain, spinal cord, heart, lungs, and kidney (i.e., essentially anywhere a disease, pathology or injury causes a change to tissue structure). For example, when used to image the brain, MRI scanners can provide information such as the location, size, orientation, and/or other details regarding the nature of a pathology (e.g., a tumour or an aneurysm), as well as the locations of the eloquent regions of the brain that should be avoided when attempting to resect a lesion. In general, MR imaging can be used to locate certain tissues and structures so that they are identified as harmful or otherwise avoided during surgery. As such, it is common for clinicians to use MRI to examine, diagnose and make surgical plans for their patients.

There are many different types of MRI techniques, including those which rely on different programmable sensitivities of MR imaging which leverage different properties of hydrogen atoms (e.g., in water and fats) as they move and interact with each other, in different ways through tissues in the body. For example, one scan type of MR imaging known as diffusion weighted imaging (DWI) measures the extent and direction of water diffusion through biological tissue. When water diffuses through tissue that has a high degree of organization, the directions available for diffusion of water molecules are unequal and distinguishable. Since the motion of water is highly sensitive to tissue cellularity and integrity, diffusion weighted magnetic resonance imaging modalities are capable of detecting abnormalities and changes to tissue structure, including conditions of illness and injury, relatively early on compared to other modalities of MR imaging and medical imaging more generally.

A category of diffusion weighted imaging known as diffusion tensor imaging (DTI) takes advantage of differences in the diffusion properties of water in different tissue microenvironments, where changes in the tissue type, organization (e.g. location of a structure), architecture (e.g. co-location of structures), and changes in directionality and the presence of barriers provide insights into the connectivity and organization of tissue in organs such as the brain. DTI is recognized in the medical imaging community as a powerful imaging modality since it has the ability to resolve structural detail on the micro scale and does not require the delivery of ionizing radiation, radioactive tracers or contrast agents.

Compared to other imaging methods which distinguish between tissue that has different density, or a response to certain wavelengths of electromagnetic radiation, DTI distinguishes between tissue that have organized structure from those that are less organized, disorganized, or homogenous in nature. For example, white matter of the brain is highly organized along white matter tracts, with myelinated axons physically restricting movement of water in the radial directions (perpendicular to the direction of the tract) and allowing diffusion in the axial direction (along the direction of the tract) in the spaces bound between the axons that make up the tract. Since tumors can change the direction and location of white matter tracts, their locations must be carefully considered when traversing healthy tissue to remove a pathology located deep in the brain. For white matter tracts in the brain and for similarly organized tissues (e.g., skeletal muscle, spinal cord, kidneys, and peripheral nerves), DTI can be used to obtain valuable insights into their microstructure and organization.

Despite showing great promise as a non-invasive medical imaging tool, current DTI systems and technologies have several technological limitations. First, it can be difficult to validate the quality of DTI data as there is oftentimes no direct way to confirm the accuracy of fiber tract reconstructions. To date, there is no industry-accepted MRI phantom instrumentation that can represent organized tissue structures with modules that have suitable properties of parameterization, precision, repeatability of manufacture, and temporal stability. As a result, it can be challenging to obtain high quality DTI images that have been validated to accurately visualize fine details within tissues. Second, DTI is highly sensitive to the subject's motion, due to small movements causing non-negligible inaccuracies in diffusion measurements. Examples of potentially disruptive motions include both bodily motions (e.g., heartbeat, breathing, blood pulse) and gross movements of the patient, which can be further complicated by factors like disease symptoms (e.g., tremor) and the patient's level of comfort. As the biomarker information in MR datasets that correspond to a disease state, or indication of a disease state, or symptom of a disease state, or an injury, such as a sub-traumatic brain injury, can be close to the unwanted or random level of background signal (e.g, as in DTI), MR imaging is said to have a low signal-to-noise ratio (SNR).

In addition, there is currently no accepted standard to assess the quality of DTI data (i.e., no good metrics of data quality). As such, poor, or unknown metrics of data quality limit the reliability and comparability of DTI sequences acquired at different times, and/or across different imaging platforms. In some cases, the baseline for data quality in a multisite MR imaging study is set by data from the poorest performing MRI system. These issues can significantly limit the reliability and efficacy of research results or studies and clinical trials performed across various imaging centers. MRI system instability and differences in baseline performance also makes repeated temporal measures difficult to compare quantitatively. This limits the potential use of current MRI systems, and its applicability to in-vivo clinical applications where knowledge of white matter structures, their integrity, and presence of abnormalities are pertinent (or germane) to good outcomes (e.g., in planning a neurosurgical intervention, where information regarding the location of a specific tract can be used to identify this structure, can describe a volume containing the structure, and a clinician may choose to avoid interacting with, traversing or removing this tissue). For example, in the case of an epilepsy surgery, a surgeon could target a specific tract to prevent the propagation of signals to prevent a grand mal seizure in a patient.

There remains a need for systems and methods for improving the data reliability of MRI methods, and more specifically diffusion weighted MR imaging modalities such as DTI. In particular, there remains a need for systems and computer-implemented methods for normalizing and quantifying temporal and imaging platform variations in DTI data. There also remains a need for systems and computer-implemented methods that are capable of validating the accuracy of DTI measurements across both time and different DTI machines.

### SUMMARY OF THE DISCLOSURE

One aspect of the invention relates to a computer-implemented method for analyzing patient data obtained from a magnetic resonance imaging (MRI) machine. The method comprises receiving at least one set of phantom data, analyzing the received phantom data to generate metrics for assessing MRI performance, and comparing the generated MRI metrics with previous MRI metrics stored in a data library to generate output data for the analysis of patient data. The phantom data may be obtained from the MRI machine scanning a phantom, such as a diffusion tensor imaging (DTI) phantom, configured for the purposes of validating accuracy of in-vivo measurements, such as DTI relevant metrics, across time and vendor.

In some embodiments, the at least one set of phantom data comprises a second set of phantom data obtained from a second MRI machine scanning the phantom. In some embodiments, the at least one set of phantom data comprises a second set of phantom data obtained from the MRI machine scanning a second phantom. In some embodiments, the at least one set of phantom data comprises a second set of phantom data obtained from a second MRI machine scanning a second phantom.

The method may involve identifying the patient data obtained from the MRI machine as validated or invalidated based on the output data. The invalidated patient data may be subsequently discarded. The method may involve generating reports based on the output data and formatting the generated reports for display in a graphical user interface. The method may involve storing the phantom data and the generated MRI metrics in the data library. The MRI metrics may include one or more of fractional anisotropy (FA), apparent diffusion coefficient (ADC), and mean diffusivity (MD). The MRI metrics may include metrics which evaluate congruence and alignment of co-registered MR datasets, metrics which evaluate alignment with a computed tomography (CT) dataset, and/or whether the spatial arrangement of features in the co-registered MR datasets are equivalent. The method may involve delivering the output data to a third party system storing the patient data.

In some embodiments, the phantom comprises one or more anisotropic diffusion modules of a well-defined filament material. In some embodiments, the phantom is a DTI phantom. The DTI phantom may contain anisotropic diffusion modules. In some embodiments, the DTI phantom comprises one or more isotropic diffusion modules. In some embodiments, the DTI phantom comprises one or more fiber networks, wherein each of the one or more networks represents a different health state of organized tissue, such as white matter. In some embodiments, the DTI phantom comprises one or more modular scaffolds, wherein each of the one or more modular scaffolds supports arrangements of fiber bundle networks. In some embodiments, DTI phantom comprises inner housing elements immersed with a matrix fluid producing biologically relevant T1 and T2 values. In some embodiments, the DTI phantom comprises directional and crossing fibers mimicking a neurological environment.

The methods described herein may be used in any one of the following applications: (a) studying efficacy of a drug for treatment of a health condition, such as a neurological condition; (b) studying effect of a drug over a period of time; (c) determination of medical treatment plans; (d) optimizing medical device and brain-computer interface (BCI) design for surgery; (e) determination of surgical site parameters for a brain-computer interface; (f) determination of placement and/or position of deep brain stimulation probe; (g) analysis of patient data to assess injury severity and/or predict recovery rates; (h) performing a retrospective on medical claims related to neurological errors; (i) analysis of patient data to determine effect of confounding factors such as previous injury, psychological assessment, depression, happiness and social integration on neuro-deficits, injury severity, care plans, recovery path, recovery time and/or rehabilitation costs; (j) assessment of the use of deep learning, artificial intelligence (AI) or machine-learning approaches for detecting disease from MRI data; (k) product development, validation and operation of MRI systems, including installation and performance checks of MRI systems and/or training and onboarding of personnel with regards to operation of MRI systems; and (l) monitoring and controlling quality metrics and performance of field deployed MRI systems.

Another aspect of the invention relates to a system for analyzing magnetic resonance imaging (MRI) patient data. The system comprises an input, a data analyzer, a data library, and an output. The input is configured to receive one or more sets of phantom data obtained from a MRI machine scanning a phantom configured for validating accuracy of in-vivo measurements across time. The data analyzer is configured to generate current MRI metrics for assessing performance of the MRI machine based on the one or more sets of phantom data. The data library is configured to store the current MRI metrics and previous MRI metrics obtained for the MRI machine. The output is configured to provide output data generated from comparisons between the current MRI metrics and the previous MRI metrics.

Additional aspects of the present invention will be apparent in view of the description which follows.

### BRIEF DESCRIPTION OF THE DRAWINGS

Features and advantages of the embodiments of the present invention will become apparent from the following detailed description, taken with reference to the appended drawings in which:

FIG. 1 is a block diagram of a magnetic resonance imaging (MRI) validation system according to one embodiment.

FIG. 2 is a flowchart of an exemplary computer-implemented method for analyzing data collected through a DTI process.

FIG. 3A is a proton density weighted image of a DTI phantom used in an experiment conducted to study features of the FIG. 1 system. FIG. 3B is a computed tomography (CT) scan of the FIG. 3A DTI phantom. FIG. 3C is a photograph of the FIG. 3A DTI phantom.

FIG. 4A is a statistical model from the experiment showing the fractional anisotropy (FA) through different vendor motion probing gradients (MPG) over time. FIG. 4B is a statistical model from the experiment showing the mean diffusivity (MD) through different vendor MPGs over time.

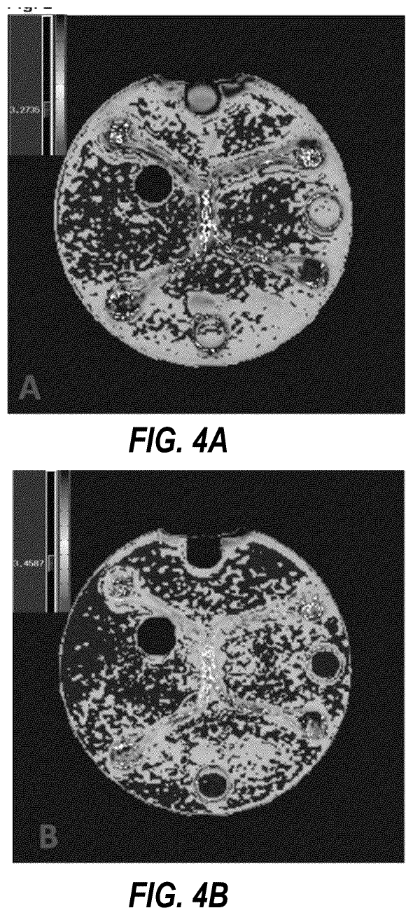

FIG. 5A is a Bonferonni corrected (p<0.05) T-score FA map through phantom fibers showing differences between a GET system and a Philips™ system used in the experiment. FIG. 5B is a Bonferonni corrected (p<0.05) T-score FA map through phantom fibers showing differences between a Philips™ system and a Siemens™ system used in the experiment. FIG. 5C is a Bonferonni corrected (p<0.05) T-score FA map through phantom fibers showing differences between a Siemens™ system and a GE™ system used in in the experiment. FIG. 5D is a Bonferonni corrected (p<0.05) T-score MD map showing differences between a GE™ system and a Philips™ system used in the experiment. FIG. 5E is a Bonferonni corrected (p<0.05) T-score MD map showing differences between a Philips™ system and a Siemens™ system used in the experiment. FIG. 5F is a Bonferonni corrected (p<0.05) T-score MD map showing differences between a Siemens™ system and a GE™ system used in in the experiment.

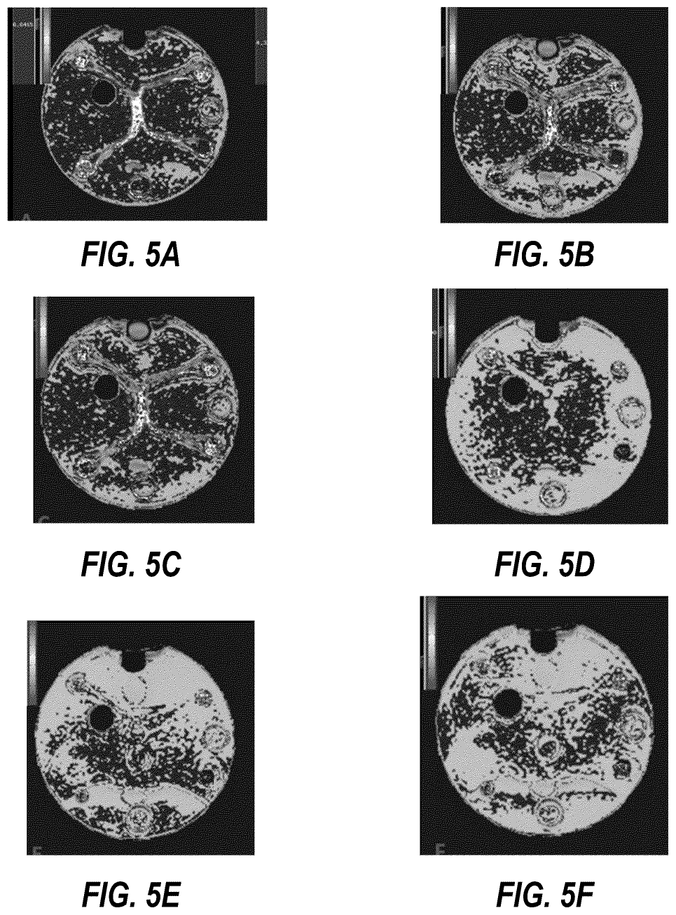

### DETAILED DESCRIPTION

The description, which follows, and the embodiments described therein, are provided by way of illustration of examples of particular embodiments of the principles of the present invention. These examples are provided for the purposes of explanation, and not limitation, of those principles and of the invention.

Aspects of the invention relate to systems, methods, and analytical tools for generating curated MRI data that can be used post-facto to assess the baseline performance of an MRI system or multiple MRI systems across different vendors (i.e., different manufacturers of MRI machines) or scanning protocols. The data can be provided to experts and be used in various applications, including for qualifying and quantifying the efficacy and clinical benefit of drug candidates, medical devices, and brain-computer interfaces. The data can provide an after the fact measure of performance for retrospectives to benefit and support clinical judgement and patient monitoring, iteration of workflows and best practices, and investigations of medical error.

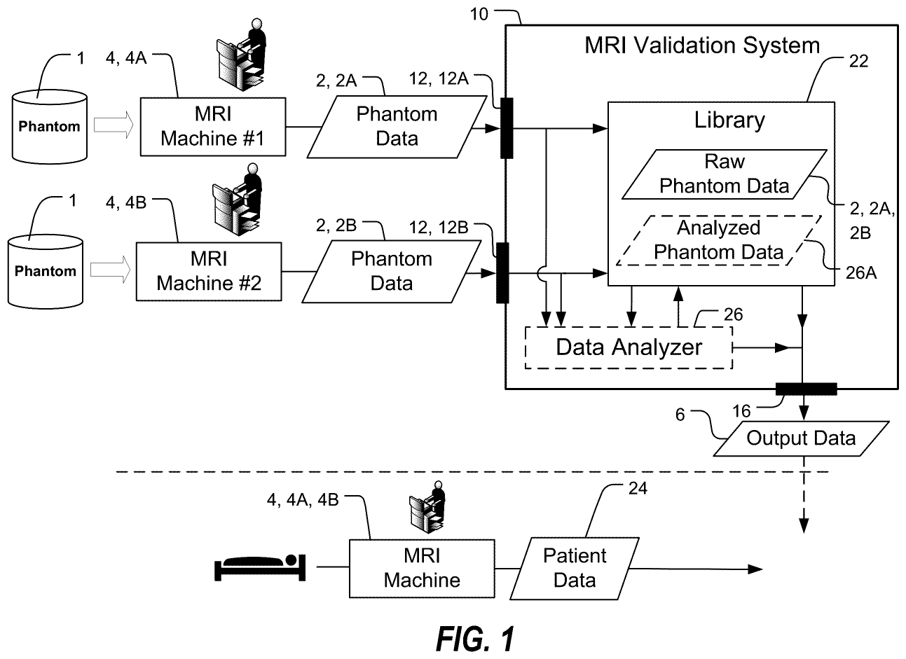

Referring now to FIG. 1, shown therein is a block diagram of a magnetic resonance imaging (MRI) validation system 10 according to an example embodiment. System 10 may be adapted to validate the quality of data acquired by different MRI machines at different points in time, including data acquired through anisotropic diffusion imaging, isotropic diffusion imaging, and/or perfusion imaging. In anisotropic diffusion imaging, the directionality of water diffusion is evaluated to visualize connectivity of the brain and other organs. In isotropic diffusion imaging, the directionality of diffusion is not accounted for, which can simulate the diffusion characteristics of bodily fluids such as cerebrospinal fluid, tissues with abnormalities, and pathologies. In perfusion imaging, changes, in oxygenation levels in tissue (e.g. in the case of a patient recovering from stroke) or the presence of a contrast agent (such as gadolinium) or other contrast agent for example, are evaluated to highlight areas with abnormal blood flow.

System 10 may be implemented through software and/or hardware, including dedicated digital processing systems executing software code or customized hardware designed to store and analyze MRI phantom data. System 10 comprises one or more inputs 12 or other means for receiving phantom data 2. In the illustrated embodiment, system 10 comprises a first input 12A for receiving phantom data 2A generated by a first MRI machine 4A, and a second input 12B for receiving phantom data 2B generated by a second MRI machine 4B. In such embodiments, system 10 may receive phantom data 2 from multiple MRI machines 4 simultaneously. Although inputs 12A and 12B are depicted as separate components in FIG. 1, this is not necessary as system 10 may alternatively comprise a shared common input for receiving phantom data 2 from various different MRI machines 4 at various different points in time. The various different MRI machine 4 may include, for example, systems manufactured by GE Healthcare, Siemens Healthineers, Philips Healthcare, Hyperfine, Canon Medical Systems, and others.

In embodiments where system 10 is implemented through hardware, inputs 12 may be provided through one or more ports that are connectable (e.g., through a wire, a cable, etc.) to a processor, computer, cloud-hosted platform, or MRI system 4 to receive data therefrom. In embodiments where system 10 is implemented through software, inputs 12, 14 may be provided through a network connection (e.g., a wired connection or a wireless connection) of the system running the software. In general, inputs 12, 14 may be provided via any suitable means for system 10 to receive phantom data 2 from an external source (e.g., database, files, web service or other service, and the like, provided via the Internet, computer, MRI system, data storage device, etc.).

As depicted in FIG. 1, phantom data 2 comprises or otherwise corresponds to data obtained from imaging an MRI phantom 1 using, for example, DTI or other modes of MRI. Preferably, the imaged MRI phantom 1 provides or otherwise comprises module representations of organised tissue and, optionally, isotropic diffusion modules. Such phantoms may be specifically designed for quality control (QC) purposes or for DTI in some cases, are usually vendor agnostic and stable over time, and may also be referred to herein as “DTI phantoms”. Various DTI phantoms may be designed for the purposes of validating accuracy of in-vivo measurements across both time and vendor. Although alternatives such as regular imaging phantoms (i.e., ones that are not a QC phantom or specifically designed for DTI) may also be used to obtain phantom data 2, these phantoms are usually not capable of quantifying temporal and imaging platform variations. In general, DTI phantoms can be integral components of an MRI quality assurance (QA) system, which may include the phantom, agreed upon reference standards, pass/fail criteria, and the method(s) of data analysis. One example of a suitable DTI phantom that can be used in the system 10 discussed herein is described in International Patent Application Publication No. WO2022077125, the contents of which are herein incorporated by reference in their entirety.

In some embodiments, phantom 1 comprises one or more anisotropic diffusion modules of a well-defined filament material that approximates the size and scale of the myelinated axons of white matter. In some embodiments, phantom 1 comprises one or more isotropic diffusion modules. In some embodiments, phantom 1 comprises one or more fiber networks, with each network representing a different health state of organized tissue. In some embodiments, phantom 1 comprises one or more modular scaffolds that support a plurality of arrangements of fiber bundle networks such that network terminus and bifurcation/intersection locations are in desired locations and orientations. In some embodiments, additional isotropic diffusion modules composed of isolated domains containing a solution with predictable ADC properties are co-located on this inner-housing system of phantom 1. In some embodiments, the inner housing elements of phantom 1 are immersed with a suitable matrix fluid producing biologically relevant T1 and T2 values.

Phantoms of the type described above can help provide a solution for temporal, multisite, and/or multivendor comparisons of DTI data. As described in more detail below, experiments have been conducted to demonstrate that reliability and comparability of data obtained across imaging platforms is possible by using DTI phantoms in connection with systems of the type described herein, thereby providing vendors with the ability to standardize their protocols. DTI phantoms can also help eliminate the logistical hurdles associated with travelling human “phantoms” and ensures appropriate calibration to the DTI sequences across vendors. This can help unlock more capabilities of DTI sequences to improve pre-surgical planning, more accurately investigate white matter pathway disruptions, improve fiber tracking, improve our understanding of human brain connectivity, and/or become a more effective imaging biomarker in a variety of neuropathologies.

Referring back to FIG. 1, phantom data 2 may, optionally, be stored in one or more databases of system 10. In some cases, this is accomplished through customized software that automatically obtains or collects MRI datasets, including phantom data 2, from various sources (e.g., MRI machines 4, computers, online databases, etc.) for the purposes of curating a data repository. The repository may be configured to include parameters such as the model of the DTI phantom, the date, the MRI system, software, and the scanning protocol. As depicted in FIG. 1, phantom data 2 may be stored in a library 22 of system 10.

Library 22 may be configured to store both raw phantom data 2 and analyzed phantom data 26A. In some embodiments, library 22 comprises a collection of phantom scans, raw phantom data 2, including raw DTI phantom data 2A, 2B, and/or analyzed phantom data 26A that track historical performance of the MR systems that were used to obtain the raw data 2, 2A, 2B. In various applications involving analysis of real-time or stored patient data 4, a baseline of MRI system performance can be inferred by, for example, querying system 10 to access the raw data 2, 2A, 2B and/or the analyzed data 26A.

As depicted in FIG. 1, an optional data analyzer 26 of system 10 may be configured to analyze or otherwise process raw phantom data 2, including raw DTI phantom data 2A, 2B, to generate analyzed phantom data 26A. The analyzed phantom data 26A may be stored in library 22 or provided directly to an external system through an output 16 of system 10. Like inputs 12, output 16 may be provided via any suitable means for system 10 to provide output data 6 to another system, module, or component for further processing or analysis. In general, the processing operations performed by data analyzer 26 may involve analysis of both raw data 2, 2A, 2B and analyzed data 26A.

By way of example, analyzed phantom data 26A may comprise or correspond to important QC or QA metrics for assessing MRI performance, such as fractional anisotropy (FA) (i.e., a unit-less value between 0 and 1 measuring the voxel-wise shape of the diffusion ellipsoid where “0” corresponds to a circle representing isotropic diffusion, “1” represents a line, and values between “0” and “1” represent ellipsoids of various configurations), apparent diffusion coefficient (ADC), and mean diffusivity (MD). The analyzed phantom data 26A may also include metrics which evaluate the alignment of co-registered MR datasets (e.g. DTI and T1, or DTI and T2, or DTI and CT) and/or whether the spatial arrangement of features in these datasets are equivalent, or the degree to which they are equivalent. Such metrics and the raw phantom data 2 collected across different MRI machines 4A, 4B, or the same MRI machine 4 at different times, may be compared to determine whether a particular set of phantom data 2 is suitably equivalent to phantom data 2 obtained in another scan.

In one exemplary application of system 10, patient data 24 may be obtained from a first MRI machine 4A at a first time point (e.g., when patient was diagnosed) and from a second MRI machine 4B at a second time point (e.g., after the patient has received treatment). Differences in performance between the first and second MRI machines 4A, 4B may be assessed with the output data 6 of system 10. For example, patient data 24 obtained from the first and second time points can then be harmonized to account for the performance differences between the first and second MRI machines 4A, 4B. In particular, the differences can be more accurately identified and assessed to understand progression of a disease, or a recovery from injury (e.g., traumatic brain injury, stroke). As another example, patient data 24 collected from an MRI machine may be provided to system 10 (e.g., in real-time) and compared with output data 6 for data quality validation purposes. Alternatively, output data 6 may be provided to the MRI machine. Patient data 24 may be compared with output data 6 and identified as either validated data (e.g., high quality data obtained from, for example, a validated MRI machine) or invalidated data (e.g., low quality data obtained from, for example, an MRI machine that has not been validated). In some cases, the invalidated data will be discarded by the MRI machine or system 10.

Patient data 24 may comprise or otherwise correspond to data obtained from imaging a patient using DTI. Although not necessary, this will typically involve imaging a head portion of the patient, since most DTI technologies have currently been adapted for the purposes of examining the brain or other organized tissues. To obtain patient data 24, a patient is imaged using one or more suitable research or clinical DTI systems.

In another exemplary application of system 10, the raw phantom data 2, 2A, 2B and/or the analyzed phantom data 26A is provided through output 16 to other systems, components, or modules for further analysis. For example, the output data 6 of system 10 may be compared to other sets of data, like the primary scan data (i.e., T1 or T2 series) taken in the same scanning session. The two datasets may then be compared to determine if they co-register such that the locations of modules are equivalently defined by both series. In addition, whether the locations are accurate can be determined in situations where the phantom information is non-symmetrical. Optionally, software may be programmed or configured to automate the extraction of various metrics from output data 6. The output data 6 may be provided to experts or clinicians through, for example, a report for further analysis. The report may, for example, be generated based on output data 6 and formatted for display in a graphical user interface (GUI) of a computer. The report may be formatted to include graphs, tables, plots, histograms, charts, etc. The output data 6 may also be provided to a third party system for storage and further analysis.

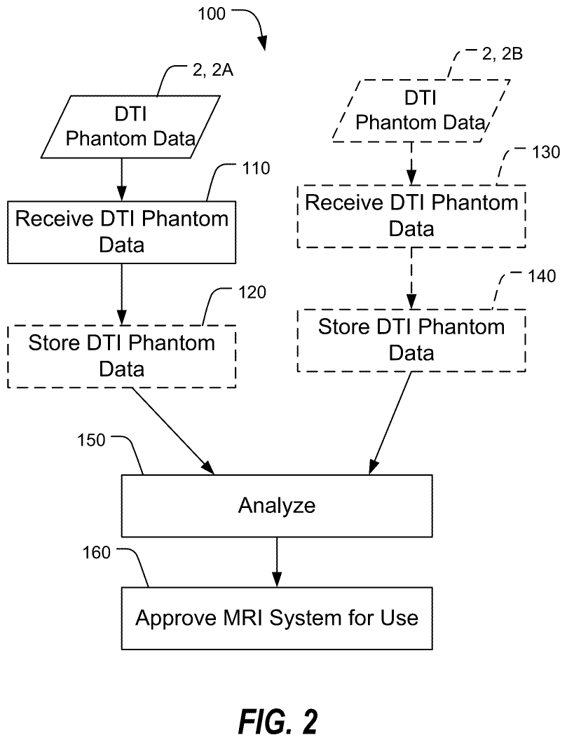

Referring now to FIG. 2, shown therein is a flowchart of an exemplary method 100 for analyzing data collected through a DTI process. Method 100 may be performed or implemented by DTI analysis system 10. Method 100 begins at step 110 where a first set of DTI phantom data 2A obtained from a first MRI machine 4A is received through, for example, input 12 of system 10. Optionally, the received DTI phantom data 2 may be stored in, for example, a DTI library 22 of system 10 at step 120. Alternatively, the received DTI phantom data 2 may be stored in a cache for immediate access by a processor or DTI data analyzer 26 of system 10. In general, steps 110 and 120 may be repeated any number of times to build a library or repository of stored DTI phantom data 2A.

Method 100 may optionally proceed to step 130 upon completion of step 110. Step 130 may be performed in parallel with, before, or upon completion of at least one instance of step 110. Step 130 comprises receiving a second set of DTI phantom data 2B obtained from a second MRI machine 4B through, for example, input 12 of system 10. In some cases, step 130 is performed after the completion of steps 110, 120. In other cases, step 130 is performed simultaneously with steps 110, 120. In other cases, step 130 is performed prior to the performance of step 110. Like the first set of received DTI phantom data 2A, the second set of received DTI phantom data 2B may, optionally, be stored in, for example, DTI library 22 of system 10 at step 140. Alternatively, the second set of received DTI phantom data 2B may be stored in a cache for immediate access by a processor or DTI data analyzer 26 of system 10. In general, whether steps 130, 140 are performed may depend on the specific application or use case of method 100.

Upon completion of step 110 and optional steps 120, 130 140, method 100 proceeds to step 150. At step 150, the DTI phantom data 2, 2A is analyzed to determine whether it is consistent with expectations and/or accurate relative to a predefined standard. Where optional step 130 is performed, the second set of DTI phantom data 2B may also be analyzed together with the first set of DTI phantom data 2A. Step 150 may be performed by DTI data analyzer 26 of system 10. If the DTI phantom data 2, 2A is determined to be accurate at step 150, then the MRI machine 4A that imaged the phantom to generate the DTI phantom data 2, 2A is approved for use at step 160.

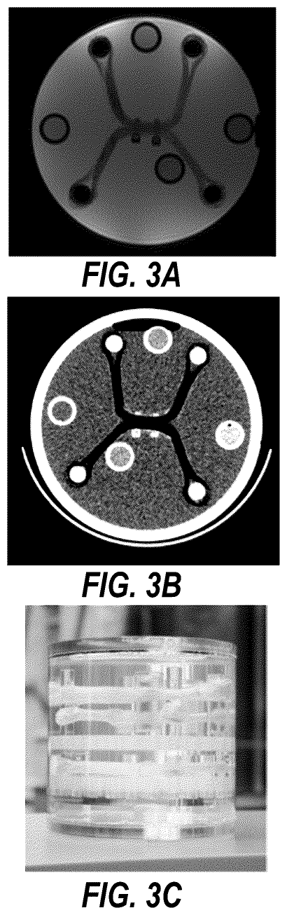

To ascertain the advantages of incorporating DTI phantom data 2A, system 10, and method 100 in the analysis of patient data 24, several experiments have been conducted. In one experiment, whether a DTI phantom could be vendor agnostic and stable over time was investigated. In the experiment, MRI systems from three (3) different vendors were used to scan a specific DTI phantom, designed to mimic a neurological environment, with multi directional fibers, crossing fibers, and appropriate brain MRI signal relaxivity values. The scanned DTI phantom contained vials of varying ADC values and also fiber bundles to mimic white matter. The DTI phantom used in the experiment is shown in FIGS. 3A-3C.

In the experiment, the DTI phantom was scanned using a research-dedicated MRI system manufactured by GE Healthcare (i.e., GE MR750 3T) and thirty-two (32) channel head coil (with software ver. 29.1, GE HealthCare). To test consistency of measurements, eight (8) separate DTI scans were performed to match the specifications on a clinical system (i.e., 32 directions, b=1000 s/mm², 4b=0 s/mm² images, 2 mm isotropic voxels, 70 slices). In the experiment, the real-time field adjustment feature of the MRI system was selected to avoid the need for a Bo map (i.e., a representation of the spatial distribution of the static magnetic field strength, which can be used to assess and correct variations like magnetic inhomogeneity).

Because motion probing gradients (MPGs) are different across vendors, identical measures were also performed using MPGs from MRI systems manufactured by Philips™ and Siemens™. All data was subsequently processed by the MRI systems to generate fractional anisotropy (FA) and mean diffusivity (MD) maps. Image co-registration was subsequently performed using the FSL command flirt using a 12-degree of freedom (df) trilinear interpolation. Lastly, to assess consistency in both time and across imaging platforms, a 2-way 3d ANOVA was performed on both FA and ADC maps, over the entire DTI phantom, using the AFNI command 3dANOVA2 with “repeat” and “vendor” as factors.

Based on false discovery rate (FDR) corrected p-values, F-statistics were calculated using a 3D 2-way ANOVA. The F-statistics showed there was no appreciable difference in either FA (i.e., see FIG. 2A) or MD (i.e., see FIG. 2B) across vendors MPGs and time. The variation in global FA values between all three MRI systems, and in data at different points in time, was 0.32%. There were no appreciable differences in fiber FA values when comparing across the different system's MPGs and across time (i.e., see FIGS. 3A-3F). Similarly, the variation of mean diffusivity (MD) across all scans was 0.0019% (i.e., see FIGS. 3A-3F). The experiment therefore indicated that DTI phantoms could be vendor agnostic and stable over time.

In addition to the exemplary aspects described above, the present invention is further described in the following example use cases, which are set forth to aid in the understanding of the invention, and should not be construed to limit in any way the scope of the invention as defined in the claims which follow thereafter.

### EXAMPLE 1—MULTI-CENTER PHARMACEUTICAL STUDY

In this example, a pharmaceutical company performs a multi-center study (e.g., an imaging biomarker study) to determine the efficacy of a drug to, for example, slow or halt the progression of a neurodegenerative condition like Alzheimer's. The pharmaceutical company performs a random controlled study by asking participants to take the drug and monitoring the participant's brain via DWI or other non-invasive diffusion MRI scanning protocols. To determine the baseline performance of each MRI system on the day of scanning each patient, the pharmaceutical company may use each MRI system to scan a phantom 1 and store the scanned results in system 10. Scans of each patient may be subsequently provided to or stored in system 10 for analysis, thereby ensuring that variations in performance are controlled and that benefits of the drug can be measured effectively.

### EXAMPLE 2—PHARMACEUTICAL STUDY OVER A TIME PERIOD

In this example, a pharmaceutical company conducts an experiment with a drug or combination of drugs and dosages that can slow the progression of a degenerative disease condition, such as a neurodegenerative disease. The pharmaceutical company can use system 10 to create various baselines at various points in time to determine the efficacy of the drug or combination of drugs and dosages.

### EXAMPLE 3—MEDICAL TREATMENT PLANS

In this example, a clinician is performing a study of patients with glioblastoma (a form of brain cancer with very poor prognosis) to determine the extent to which an aggressive approach to resection of tissue is correlated to improved outcomes. The clinician can use system 10 to create a baseline of MR performance, and with this information, optionally set standards and approaches to defining tumour perimeters for resection that optimize patient outcomes from surgery.

### EXAMPLE 4—TUMOUR RESECTION

In this example, a study of patients with deep-seated tumours requiring resection is performed to determine the best surgical trajectory through healthy tissue to perform this resection. The determination is based on MRI information showing the connectivity of white matter and eloquent tracts in the brain of the patients. System 10 can be used to establish a control (i.e., by receiving and storing DTI phantom data 2) to improve the quality of the MRI information. The results of the study may be used by a medical device company to optimize their instruments to support creation of surgical corridors through healthy tissue such that their instruments have optimal parameters (e.g., the necessary length, diameter etc.) to best support the requirements of these surgeries, and by clinicians and hospital staff when selecting instrumentation for an intervention and surgical corridors for resection.

### EXAMPLE 5—SURGICAL SITE PARAMETERS

In this example, a company developing a brain-computer interface (BCI) wishes to set parameters such as size and location for a customer cohort. The company uses MRI scans of patients in order to determine the ideal location to place the BCI. The company also uses MRI scans of the patients to determine the connectivity of the brain and thus locate the optimal position to attach the device to clinically benefit the patient. The company can use system 10 to help determine surgical site parameters such as location, size of incision, burr-hole, craniotomy for optimal treatment and patient benefit and measure the efficacy of BCI by design and/or location, and the health state of the tissue connected to the BCI.

### EXAMPLE 6—DEEP BRAIN SIMULATION

In this example, a medical device company is testing a deep brain stimulation probe to minimize tremor in patients. Through an imaging biomarker study, the company wishes to correlate both placement and optimal position of the medical device probe with clinical benefit and improvement in the patient's symptoms. By imaging a phantom 1 and storing various sets of patient data 4 in system 10 for analysis, the study is able to compare the patient data and quantify the distance from the position of the probe in and near key structures and nerve bundles that align with the benefit. Imaging phantom 1 with a medical device, or component of a medical device, also allows the company and its representatives to better predict, qualify and quantify effects and changes in MR data properties as a result of medical device placement. This example could also apply to Class 3 medical devices in general.

### EXAMPLE 7—INSURANCE

In this example, an insurance company is looking to best predict recovery path and required time for patients with a traumatic or sub-traumatic brain injury (TBI) in order to determine and support clinical costings and business metrics. The insurance company performs a multi-site study following a number of patients with TBI in order to assess these parameters which determine business costs and market values. The insurance company notes that patients are often diagnosed at locations (e.g. an ICU) that are different from the locations used for follow-on medical imaging and monitoring of the condition. In these types of situations, the insurance company can use system 10 to create a control group at the various locations in order to better analyze patient data, assess injury severity, and determine differences and trends in the recovery rates. The insurance company can also correlate the extent of disruption of white matter, and changes during the recovery to outcomes.

### EXAMPLE 8—RETROSPECTIVE INSURANCE INVESTIGATION

In this example, an insurance carrier is performing a retrospective on medical claims related to neurosurgical errors. It can access system 10 to store MRI patient data 4A as well as DTI phantom data 2A to understand the MR systems' performance and the quality of the patient data. Optionally, the insurance carrier can also access data libraries of system 10 to see the historical record of the MR system performance. The insurance carrier can use system 10 to evaluate the MR data of the patient to determine if patient injuries or poor intervention outcome can be attributed to a medical error. The insurance carrier can then use this information to determine the carrying costs for institutions and specific surgeons based on MRI data accuracy, rates of error, and error type. Optionally this information can be shared with hospital information systems to improve best practices and standard of care.

### EXAMPLE 9—MILITARY STUDY

In this example, a body, company, non-governmental organization, or other organization with interest or responsibility regarding duty of care for active and formerly active military personnel performs a study of military personnel that have experienced or have a suspected blast-induced neuro-trauma event. Through this study, the military body wishes to determine the effect of confounding factors (e.g., previous injury, other medical data such as psychological assessment, depression, happiness and social integration, etc.) to predict neuro-deficits, injury severity, optimal care plans of affected individuals, recovery path, recovery time, and costs of rehabilitation. The body can use system 10 to analyze data from different military personnel to determine the effect of various confounding factors.

### EXAMPLE 10—ARTIFICIAL INTELLIGENCE/MACHINE LEARNING

In this example, a research team is performing a study to see if a deep learning, artificial intelligence, or machine-learning approach can be used to detect disease from MRI data. For each patient in the cohort, a scan of a phantom 1 may be taken and stored in system 10 to baseline the performance of the MR system and confirm that the quality of the data used to train the algorithm is above a threshold/minimum quality standard. Comparisons of patient data 4 at various subsequent points in time, controlled with the stored DTI phantom data 2A, can be used to detect subtle changes and trends to biomarkers that correlate with onset and/or progression of disease.

### EXAMPLE 11—MRI MANUFACTURING

In this example. an MRI system manufacturer used the historical performance of the MR system (as determined by the phantom data and trends within this data) to determine and/or predict service call necessity, deployment of field staff and resources including technical, marketing and sales efforts with regards to servicing, replacing field deployed systems, and confirming correct installation, training or onboarding of personnel.

The examples and corresponding diagrams used herein are for illustrative purposes only. Different configurations and terminology can be used without departing from the principles expressed herein.

Although the invention has been described with reference to certain specific embodiments, various modifications thereof will be apparent to those skilled in the art without departing from the scope of the invention. The scope of the claims should not be limited by the illustrative embodiments set forth in the examples, but should be given the broadest interpretation consistent with the description as a whole. For example, various features are described herein as being present in “some embodiments”. Such features are not mandatory and may not be present in all embodiments. Embodiments of the invention may include zero, any one or any combination of two or more of such features. This is limited only to the extent that certain ones of such features are incompatible with other ones of such features in the sense that it would be impossible for a person of ordinary skill in the art to construct a practical embodiment that combines such incompatible features. Consequently, the description that “some embodiments” possess feature A and “some embodiments” possess feature B should be interpreted as an express indication that the inventor also contemplate embodiments which combine features A and B (unless the description states otherwise or features A and B are fundamentally incompatible).

1. A computer-implemented method for analyzing patient data obtained from a magnetic resonance imaging (MRI) machine, the method comprising:
receiving at least one set of phantom data, the at least one set of phantom data comprising a first set of phantom data obtained from the MRI machine scanning a phantom configured for the purposes of validating accuracy of in-vivo measurements across time;
analyzing the at least one set of phantom data for assessing MRI performance;
comparing the analyzed phantom data with metrics for the MRI machine stored in a data library; and
based on the comparison, generating output data for the analysis of the patient data.

2. The method of claim 1, comprising identifying the patient data obtained from the MRI machine as validated or invalidated based on the output data, and discarding the invalidated patient data.

3. The method of claim 1, wherein the at least one set of phantom data comprises a second set of phantom data obtained from a second MRI machine scanning the phantom.

4. The method of claim 1, wherein the at least one set of phantom data comprises a second set of phantom data obtained from the MRI machine scanning a second phantom.

5. The method of claim 1, wherein the at least one set of phantom data comprises a second set of phantom data obtained from a second MRI machine scanning a second phantom.

6. The method of claim 1, comprising storing the at least one set of phantom data and the analyzed phantom data in a data library.

7. The method of claim 1, comprising generating one or more reports based on the output data and formatting the one or more generated reports for display in a graphical user interface.

8. The method of claim 1, comprising delivering the output data to a third party system storing the patient data.

9. The method of claim 1, wherein the analyzed phantom data includes one or more of fractional anisotropy (FA), apparent diffusion coefficient (ADC), and mean diffusivity (MD).

10. The method of claim 1, wherein the analyzed phantom data includes one or more of:
metrics which evaluate congruence and alignment of co-registered MR datasets, metrics which evaluate alignment with a computed tomography (CT) dataset, and metrics which evaluate whether the spatial arrangement of features in the co-registered MR datasets are equivalent.

11. The method of claim 1, wherein the phantom comprises an anisotropic diffusion module of a well-defined filament material.

12. The method of claim 1, wherein the phantom comprises a diffusion tensor imaging (DTI) phantom.

13. The method of claim 12, wherein the DTI phantom comprises an isotropic diffusion module.

14. The method of claim 12, wherein the DTI phantom comprises one or more fiber networks, wherein each of the one or more networks represents a different health state of organized tissue.

15. The method of claim 12, wherein the DTI phantom comprises one or more modular scaffolds, wherein each of the one or more modular scaffolds supports arrangements of fiber bundle networks.

16. The method of claim 12, wherein the DTI phantom comprises a plurality of inner housing elements immersed with a matrix fluid producing biologically relevant T1 and T2 values.

17. The method of claim 12, wherein the DTI phantom comprises a plurality of directional and crossing fibers mimicking a neurological environment.

18. The method of claim 1, wherein the output data is used for one or more of the following applications:
a) studying efficacy of a drug for treatment of a neurological condition;
b) studying effect of a drug over a period of time;
c) determination of medical treatment plans;
d) optimizing medical device and brain-computer interface (BCI) design for implant or surgery;
e) determination of surgical site parameters for a brain-computer interface;
f) determination of placement and/or position of deep brain stimulation probe;
g) analysis of patient data to assess injury severity and/or predict recovery rates;
h) performing a retrospective on medical claims related to neurological errors;
i) analysis of patient data to determine effect of confounding factors such as previous injury, psychological assessment, depression, happiness and social integration on neuro-deficits, injury severity, care plans, recovery path, recovery time and rehabilitation costs;
j) assessment of the use of deep learning, artificial intelligence (Al) or machine-learning approach for detecting disease from MRI data;
k) product development, validation and operation of MRI systems, including installation and performance checks of MRI systems and/or training and onboarding of personnel with regards to operation of MRI systems; and
l) monitoring and controlling quality metrics and performance of field deployed MRI systems.

19. A computer-readable memory having computer-executable instructions recorded thereon that when executed by a computer cause the computer to perform the method as defined in claim 1.

20. A system for analyzing magnetic resonance imaging (MRI) patient data, the system comprising:
an input configured to receive one or more sets of phantom data obtained from a MRI machine scanning a phantom configured for validating accuracy of in-vivo measurements across time;
a data analyzer configured to generate analyzed phantom data for assessing performance of the MRI machine based on the one or more sets of phantom data;
a data library configured to store the analyzed phantom data and metrics for the MRI machine; and
an output configured to provide output data,
wherein the output data is generated from comparisons between the analyzed phantom data and the metrics for the MRI machine.
